(19) United States
(12) Patent Application Publication
(10) Pub. No.: US 2017/0172704 A1
(43) Pub. Date: Jun. 22, 2017

(54) DENTAL BUR

(71) Applicant: Neo-Flo, Inc., Kennesaw, GA (US)

(72) Inventors: Perry L. Parke, Marietta, GA (US); Richard Muller, Gloucester (GB)

(73) Assignee: Neo-Flo, Inc., Kennesaw, GA (US)

(21) Appl. No.: 14/976,752

(22) Filed: Dec. 21, 2015

(51) Int. Cl. A61C 3/02; A61B 17/16

(52) U.S. Cl. CPC A61C 3/02; A61B 17/1615

(57) ABSTRACT

A dental bur (10) is disclosed having an elongated shank (11) which extends to a cutting head (12). The shank includes a gripping portion (14), a tapered transition portion (15), and a neck portion (16). The diameter of the neck portion (D1) is generally the same as the diameter of the cutting head (D2). The cutting head includes six helical cutting flutes (20) extending from the neck portion (16) to a tip (18). Each cutting flute has a top cutting edge (21) and a series of generally rectangular shaped cross-cut notches. The rectangular shaped cutting notches are formed by a generally flat floor (25) and two oppositely disposed sidewalls (26). The floor forms a floor cutting edge (28) while the sidewalls form sidewall cutting edges (29). The intersection of the floor cutting edge with the sidewall cutting edges form two interior cutting corners (30).

### TECHNICAL FIELD

This invention relates to dental burs, and particularly to dental burs having helical cutting blades.

### BACKGROUND OF THE INVENTION

Dental instruments are utilized to remove the decayed portion of a tooth in preparation for filling. The most common method of removing the decayed material is to form a hole in the decayed portion with the use of a rotary drill and drill bit. The tooth material may be further removed with the use of a dental bur or burr.

Today's dental burs have a multitude of designs and configurations designed for specific uses. Many of these burs have cutting blades or flutes which are used to cut away a portion of the tooth through the sideways motion of the bur. Some of these types of burs have flutes which are helical. To aid in the cutting aspect of the flutes, some bur flutes have V-shaped or semi-circular shaped notches, sometimes referred to as cross-cuts, formed horizontally across the flutes, as shown in U.S. Pat. Nos. 418,108; 4,284,406 and 5,261,818. These v-shaped or semi-circular shaped cross-cuts may improve the cutting ability of the drill by cutting the tooth into smaller pieces, however, it is believed that they do not optimize the cutting ability of a dental bur.

Another problem associated with today's dental burs is that the cutting head which includes the helical flutes is larger in diameter than the neck extending directly from the cutting head. The difference in the sizing of the neck may cause difficulty or damage when drilling into the tooth a distance longer that the length of the cutting head. For should the cutting head be drilled past the outer surface of the tooth, the cutting head may cause damage as it's direction is reversed and the upper extent of the cutting head encounters the sidewall of the drill hole.

The material which comprises the dental bur is also very important as burs made of a tungsten carbide are harder and more durable than other metals. However, their physical properties are such that they are brittle. Other burs combine the strength of a stainless steel shank with a tungsten carbide head welded to the shank. These burs have a potential problem of breaking or fracturing at the weld.

Accordingly, it is seen that a need remains for a dental bur which enables a more efficient cutting of the tooth, especially if it is drilled into the tooth a length greater than the length of the cutting head. It is to the provision of such therefore that the present invention is primarily directed.

### SUMMARY OF THE INVENTION

A dental bur comprises a shank having a gripping portion and a neck portion extending from the gripping portion, and a cutting head extending from the neck portion opposite the gripping portion. The cutting head has a plurality of helical cutting flutes with a plurality of generally rectangular shaped cross-cut notches.

In another form of the invention, a dental bur comprises a cutting head having a plurality of helical cutting flutes and an end portion with a select first diameter. The dental bur also has a shank having a gripping portion and a neck portion extending between the gripping portion and the end portion of the cutting head. The neck portion has a select second diameter generally equal to the select first diameter of the end portion of the cutting head.

### BRIEF DESCRIPTION OF THE DRAWING

FIG. 1 is a side view of a prior art dental bur.

FIG. 2 is a side view of a dental bur embodying principles of the invention in a preferred form.

FIG. 3 is an end view of the dental bur of FIG. 2.

FIG. 4 is an enlarged portion of the dental bur of FIG. 1, showing a cross-cut notch in more detail.

FIG. 5 is a chart showing the breaking force for the dental bur of FIG. 1 compared with six other burs.

### DETAILED DESCRIPTION

With reference next to the drawings, there is shown a dental bur 10 embodying principles of the invention in a preferred form. The dental bur 10 is made of a metal material, preferably a tungsten carbide, and is configured to be used in conjunction with a rotary dental drill.

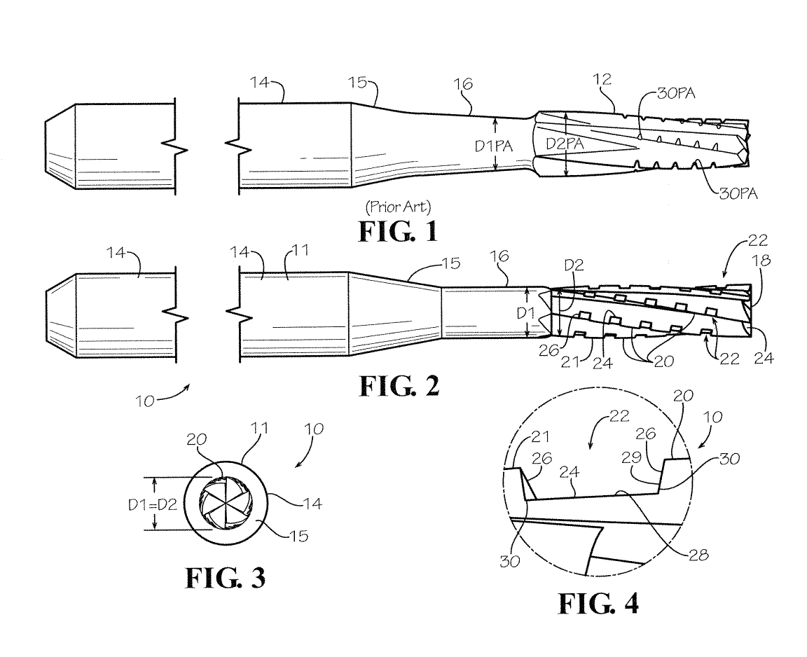

The dental bur 10 has an elongated shaft or shank 11 which extends to a cutting head 12. The shank 11 includes a gripping portion 14, which is coupled to the dental drill, a tapered transition portion 15, and a neck portion 16. The neck portion 16 extends to the cutting head 12. It should be noted that the diameter of the neck portion D1 adjacent the cutting head 12 is generally the same as the diameter of the cutting head D2 adjacent the neck portion, i.e., the diameter of the cutting head D2 and neck portion D1 are generally the same at the transition point therebetween. This is very different from helical fluted cutting heads of the prior art, shown in FIG. 1, wherein the cutting head diameter D2PA is larger than the neck diameter D1PA.

The cutting head 12 is slightly tapered toward the tip 18 and includes six helical cutting flutes 20 extending from the neck portion 16 to the tip 18. Each cutting flute 20 has a top cutting edge 21 and a series of generally rectangular shaped cutting notches, cross-cut notches, or cross-cuts 21, the embodiment shown in the drawings include five cross-cuts 21, but the number of such cross-cuts may be different according to the length of the cutting head and its desired cutting characteristics. The tip end of each cutting flute 20 is also angled so as to form a series of tip cutting edge 24 which extend from the top cutting edges 21. The rectangular shaped cutting notches are formed by a generally flat floor 25 and two oppositely disposed sidewalls 26 extending generally normal (preferably within +/−17.5 degrees, for a total offset between walls to be no more that 35 degrees) to the floor 25. The floor 25 forms a floor cutting edge 28 while the sidewalls 26 form sidewall cutting edges 29. The intersection of the floor cutting edge 28 with the sidewall cutting edges 29 form two inside or interior cutting corners 30.

It is believed that the generally rectangular shaped notch provides for a better cutting of the tooth compared to a V-shaped or semi-circular shaped notch. It is believed that the reason for the improved cutting ability is that the more upright angle of the sidewalls 26 of the rectangular shaped notches creates a better cutting edge. This configuration removes a larger portion of the tooth, distributes the cutting forces over a larger cutting surface, and decreases the stress concentration point found in the V-shaped notches. Furthermore, the rectangular shaped notches improves the cutting capabilities over time because as the bur wears with use the rectangular shaped notches still provides two cutting corners 30, as opposed to the one cutting corner 30PA of the prior art V-shaped notch.

The neck portion 16 being the same size (diameter) as the cutting head 12 also provides an advantage over the configuration of the prior art shown in FIG. 1. Having the neck portion the same size as the cutting head allows the bur to be pushed laterally without causing a tilting of the bur when the cutting head has been pushed past the outer surface of the tooth, as the neck will abut the tooth material in the same manner as the cutting head. The neck having the same diameter as the cutting head also allows the operator to better estimate the depth of the cutting head once it has past the outer surface of the tooth and is therefore obscured from view. This configuration also allows for the formation of a larger neck to increase the strength of the bur and reduce the concentration point between the smaller neck and cutting head of the prior art where most bur fractures occur.

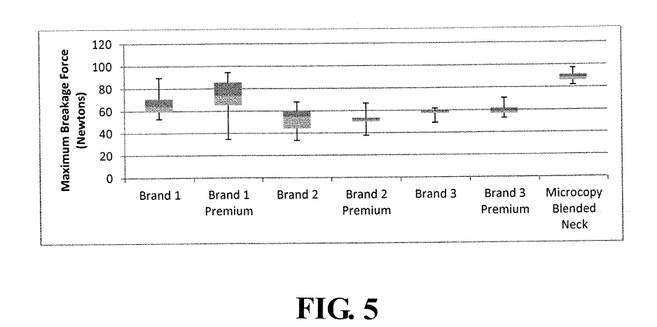

Testing was conducted on the inventive bur design compared to six other burs (each test repeated 15 times per bur), the results are shown in FIG. 5. The purpose of the test is to show the superior breaking capabilities of the inventive dental bur. The test comparison shows the breakage strengths, or maximum amount of force required to break the bur. The test was conducted by inserting a bur into a fixture the length of a standard drill hand piece. A force is then applied perpendicularly to the head and the maximum force is measured and recorded prior to breakage. The testing was repeated for each of the presently best selling tungsten carbide burs. Brand 1, Brand 1 Premium, Brand 2, and Brand 2 Premium are burs having a stainless steel shaft and a larger tungsten carbide head welded to the shaft. Brand 3 and Brand 3 Premium are burs made completely of tungsten carbide with a neck smaller in diameter than the diameter of the head.

The results of the test show that the inventive bur of the present invention had a breakage force (M=88.8 Newtons, SD=4.33 Newtons) which had a narrow range of average breaking forces (similar to a mode) with a boundary range extending between just over 80 Newtons to just under 100 Newtons. The chart illustrates that the inventive bur's boundary range was smaller and larger than the other burs in the comparison. It should be noted that while Brand 1 and Brand 1 Premium's boundary range included a portion of the inventive bur's boundary range, their boundary range was much larger extending from the high thirties to the mid nineties, also their average range was much lower. Overall, the inventive bur on average had a 50% higher breakage force than all competitors premium and regular carbide burs. The test results show that the inventive bur made of a tungsten carbide with a shaft diameter the same as the head diameter minimizes breakage, which lessens the risk to a patient of aspirating on a broken piece of a bur or other complications associated with broken burs.

It should be understood that the term generally rectangular shaped cutting notches is not intended to be limited to exactly a rectangular geometric shape, as the sidewalls may be of different heights, the floor may not be the same length as the sidewalls, and the angle between the floor and a sidewall may not be exactly 90 degrees. The term generally rectangular shaped is intend to represent a shape having a generally flat floor and two sidewalls extending from the flat floor. The term generally rectangular shaped is therefore intended to include a notch shape which may be generally square, rhombus or trapezoidal in shape.

It should also be understood that the term helical blade or flute is intended to include blades which are not perfectly helical in the geometric sense. The term helical blades is intended to also includes blades wherein only a portion of the blade is helical or spiral in configuration, blades which may be considered curved rather than helical, blades wherein the diameter of the “helix” changes (tapered), or blades which may be considered spiral.

It thus is seen that a dental bur for cutting a tooth is now provided which overcomes problems associated with such devices of the prior art. While this invention has been described in detail with particular references to the preferred embodiments thereof, it should be understood that many modifications, additions and deletions, in addition to those expressly recited, may be made thereto without departure from the spirit and scope of the invention.

1. A dental bur comprising,
a shank having a gripping portion and a neck portion extending from said gripping portion, and
a cutting head extending from said neck portion opposite said gripping portion, said cutting head having a plurality of helical cutting flutes, each said helical cutting flute having a plurality of generally rectangular shaped cross-cut notches.

2. The dental bur of claim 1 wherein said neck portion has a select first diameter and wherein said cutting head has an end coupled directly to said neck portion having a select second diameter generally equal to said select first diameter of said neck portion.

3. A dental bur comprising,
a shank, and
a cutting head extending from said shank, said cutting head having a plurality of helical cutting flutes having top cutting edges, each said helical cutting flute having a plurality of cross-cut notches, each said cross-cut notch having a generally flat floor and a pair of oppositely disposed sidewalls extending from said floor to said top cutting edge of said flute.

4. The dental bur of claim 3 wherein said sidewalk extend generally normal from said floor.

5. The dental bur of claim 3 wherein said shank has a gripping portion and a neck portion, and wherein said neck portion has a select first diameter and wherein said cutting head has an end coupled directly to said neck portion having a select second diameter generally equal to said select first diameter of said neck portion.

6. The dental bur of claim 3 wherein the diameter of said cutting head does not exceed the diameter of any portion of said shank.

7. A dental bur comprising,
a cutting head having a plurality of helical cutting flutes, said cutting head having an end portion which includes said helical cutting flutes with a select first diameter, and
a shank having a gripping portion and a neck portion extending between said gripping portion and said end portion of said cutting head, said neck portion having a select second diameter generally equal to said select first diameter of said end portion of said cutting head.

8. The dental bur of claim 7 wherein each said helical cutting flutes includes a plurality of generally rectangular shaped cross-cut notches.

9. The dental bur of claim 7 wherein each said helical cutting flutes has top cutting edges and a plurality of cross-cut notches extending from said top cutting edge, each said cross-cut notch has a generally flat floor and a pair of oppositely disposed sidewalls extending from said floor to said top cutting edge of said flute.
